(19) United States
(12) Patent Application Publication
(10) Pub. No.: US 2014/0159912 A1
(43) Pub. Date: Jun. 12, 2014

(54) BIOMEDICAL MONITOR FOR SMARTPHONE

(71) Applicant: Jacob Fraden, San Diego, CA (US)

(72) Inventor: Jacob Fraden, San Diego, CA (US)

(21) Appl. No.: 13/710,361

(22) Filed: Dec. 10, 2012

(51) Int. Cl. A61B 5/00

(52) U.S. Cl. CPC A61B 5/002
USPC 340/870.02

(57) ABSTRACT

A biomedical device for continuous or intermittent monitoring of vital signs, such a arterial blood pressure, pulse oxymetry, etc., comprises two components connected by a wireless link. The first component is an electronic bracelet attached to a patient, while the second one is a smartphone that controls the first component and receives from it biomedical signals. The bracelet carries various sensors and actuators to enable and acquiring medical signals. The smartphone has an app that commands the bracelet and then receives and processes data and takes further actions, like enabling an alarm, plotting data, calling an emergency service or doctor office.

### FIELD OF INVENTION

This invention relates to mobile communication devices, or more specifically, to mobile communication devices capable of collecting and monitoring external signals.

### DESCRIPTION OF PRIOR ART

Nowadays in their versatility, smart telephones resemble a Swiss Army Knife—a multi-function and multi-purpose device. Most wireless communication devices (cellular or mobile telephones, e.g.) incorporate additional non-communication features, such as imaging (photo and video), personal planners, games, navigation, etc. There are numerous inventions that attempt to include additional features for measurement and/or monitoring external signals such as temperature and air pressure. Especially of interest for practical applications are medical uses of smartphones for the purpose of patient monitoring, self-diagnostic and treatment. Certain medical monitoring detectors can be imbedded directly into a smartphone and be an integral part of such. An example is a non-contact infrared medical thermometer being part of a smart phone as taught by the U.S. Pat. No. 8,275,413 issued to Fraden et al., that is incorporated herein as a reference. Yet, many other biomedical signs, for example, arterial blood pressure, EKG, blood glucose and others, require more intimate interfaces between the sensors and patient. In other words, these vital signs can't be measured remotely and require a direct physical contact with the patient body surface. Incorporating the specialized sensors on or inside a smartphone is impractical as it would impair other functions of the phone, making it large and expensive. Thus, as currently known in the art, a mobile communication device (smartphone, e.g.) serves only as an information link between a stand-alone medical monitor and external devices, either local or remote. An example of such an approach is the U.S. Patent Publication No. 2007/0073173 A1 issued to Lam et al., being incorporated herein as a reference. This publication teaches a wrist blood pressure monitor that via a cable is connected to an external computer or cell phone for transmission of the collected information. Thus, a mobile phone is not part of the data acquisition process and functions independently of such process as a mere communication channel.

Any stand-alone medical monitor can send its output signals via a mobile communication device (cell phone, e.g.). Clearly, this is well known in the art. What is not known is combined system where one component can't function without the other. Further, it makes a practical sense to interface a cell phone only with portable medical monitors that can be carried by or worn on the patient body. Otherwise advantages of a handheld smartphone (small size, versatility, multiple purposes, etc.) become irrelevant. Modern progress in electronics and packaging resulted in a significant size and weight reduction of many medical devices. Examples are the wrist blood pressure monitors, glucometers, audiometers, body impedance meters, and many others. Even mass spectrometers for the chemical analysis of bodily fluids have been produced by using MEMS processes. These vital signs are of interest for the patient monitoring.

In several practical applications, such as sport medicine, clinical monitoring of moving patients, elderly care and several others, it is desirable for patients to carry vital sign monitors on their bodies and collect medical data with little or no interferences with other personal activities. Many of these patients conduct active way of life and usually carry with them a mobile phone. Therefore, it is desirable to achieve a synergy between the smartphone and a portable medical data acquisition system. For a better efficiency, synergy should go beyond a mere data communication by a phone and preferably make a smartphone an integral part of the monitor.

Use of a mobile phone for transmitting data from the monitoring system comprising an implantable sensor device is taught by the U.S. Pat. No. 8,265,556 issued to Tekin, et al. Also, a mobile phone can be used for controlling functionality of medical devices as exemplified by U.S. Pat. No. 8,015,972 issued to Pirzada. A near-range wireless communication between medical devices is known in art as exemplified by the U.S. Pat. No. 7,565,132 issued to Orbach. These patents are incorporated herein as references.

Small medical monitors are known in art. An example is a wrist blood pressure monitor that is fabricated in form of a bracelet and being totally self-contained. This device is exemplified by the U.S. Pat. Nos. 5,640,964 issued to Archibald et al. and No. 7083573 issued to Yamakoshi et al., such patents being incorporated herein as references. A wearable patient monitoring system is exemplified by the U.S. Patent Publication No. 2009/0204013 A1 issued to Mühlsteff et al., such publications being incorporated herein as a reference. A cuffless wrist blood pressure monitor operating with a simple cell phone to transmit medical information via Internet is disclosed in US 2005/0228300 A1 issued to Jaime et al. The disclosure being incorporated herein as a reference.

Thus, it is an object of the present invention to provide a wearable device that combines functions of a smartphone and medical data acquisition system.

It is another object of the present invention to make a small wrist bracelet containing medical sensors and being controlled by a personal mobile communication device;

Further and additional objects are apparent from the following discussion of the present invention and the preferred embodiments.

### SUMMARY OF THE INVENTION

A device wearable on the outside of the patient body or body part, for example such device is a bracelet containing medical sensors and actuators. The wearable device comprises a module for a near-range wireless link with a mobile communication device (smartphone or tablet, e.g.). Main function of the bracelet is to collect and condition medical data and then transmit them wirelessly to a smartphone. The smartphone stores and uses a pre-loaded software app capable of controlling the wearable sensors and actuators in the bracelet and to process, analyze and output medical data, such as arterial blood pressure, heart rate, blood oxygenation, and others received from the bracelet. The app is capable of presenting the result of the data processing on the smartphone output means, such as a display.

### BRIEF DESCRIPTION OF DRAWINGS

FIG. 1 illustrates a block-diagram of a sensing bracelet communicating with a smartphone.

FIG. 2 is a block diagram of a bracelet containing an arterial blood pressure monitor with an inflatable cuff (bladder) and an arbitrary medical detector for other vital signs.

FIG. 3 depicts a cross-sectional view of a bracelet with an inflatable blood pressure bladder installed on a patient wrist.

FIG. 4 is a simplified flow-chart of an app for measuring blood pressure (BP): systolic (SYS), diastolic (DIA) pressures and heart rate (HR).

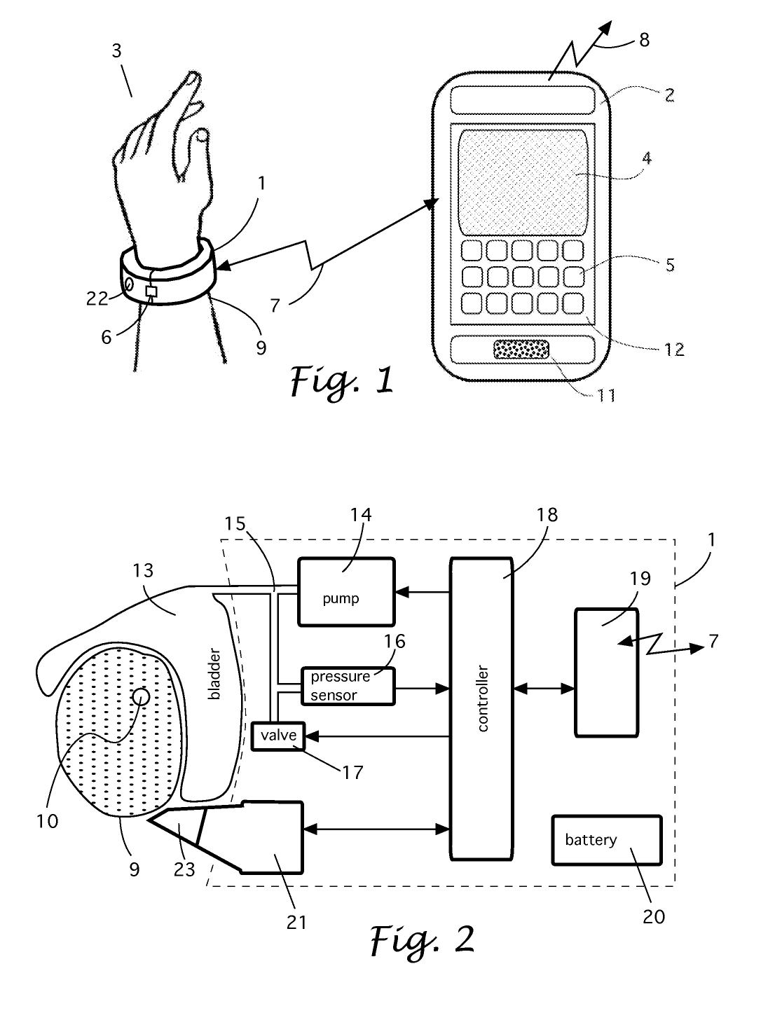

### PARTS LIST FOR FIGS. 1-4

| | |
|---|---|
| 1 | bracelet |
| 2 | mobile phone |
| 3 | patient hand |
| 4 | display |
| 5 | keypad |
| 6 | latch |
| 7 | radio signal |
| 8 | wireless signal |
| 9 | wrist |
| 10 | artery |
| 11 | speaker |
| 12 | control panel |
| 13 | bladder |
| 14 | air pump |
| 15 | tubing |
| 16 | pressure sensor |
| 17 | air valve |
| 18 | controller |
| 19 | communication module |
| 20 | battery |
| 21 | medical detector |
| 22 | indicating light |
| 23 | optical elements |
| 24 | electronic module |
| 25 | first cushion |
| 26 | second cushion |
| 27 | back shell |
| 28 | front shell |
| 29 | Velcro latch |
| 30 | bones |
| 31 | pivot |

### DESCRIPTION OF PREFERRED EMBODIMENTS

Refer to FIG. 1 that shows a patient hand 3. A lightweight bracelet 1 is positioned on a wrist 9 and secured on it by a latch 6. Alternatively, the bracelet may be positioned on an ankle of the patient. The bracelet 1, among other components, comprises a module for a near-range wireless communications with an external device, for example, a smartphone 2 or tablet. A near-range means here is a distance from the bracelet up to 2 m—a sufficient practical range for the present invention. An example of a popular near-range communication is a Bluetooth™ protocol that communicates at a range up to 30 m. The radio signal 7 carries a bidirectional information between the bracelet 1 and smartphone 2. Smartphone 2 has a conventional module for sending wireless signals 8 to remote re-transmission and communication stations. The phone 2 also has conventional human interface features, such as display 4 on the control panel 12, keypad 5 of any kind, and speaker 11. The phone 2 has pre-installed a software app for communicating with the bracelet 1, sensing to it various commands and processing the received medical data. The app should be specific for a particular type of bracelet 1 and the sensors it carries.

With respect to functionality, functions of the bracelet 1 and phone 2 are clearly separated in order to optimize their respective sizes, complexity and enhance efficiency. The bracelet functions are generally should be limited to data acquisition and transmission. Thus the bracelet (sensing device) is one part of the combined system of a bracelet+phone. The bracelet collects medical signals, conditions and sends them to the processing part of the system which is situated in a smartphone 2 and controlled by the app. As a result, bracelet 1 doesn't need a complex processor, signal processing software, display, speaker or other human interface components that normally would be required in a stand-alone monitor. These functions are shifted to the smartphone 2 that already has such component shared with other phone functions. The bracelet 1 may need some kind of patient signaling components, for example, the indicating lights 22 to signal the bracelet operating conditions. Examples of the conditions are power on/off, wrong placement on a wrist, closed/open latch 6, etc. Naturally, besides the indicating lights 22, other types of a feedback may be employed, for example a beeper.

FIG. 2 illustrates a block-diagram of one embodiment of a bracelet 1 for monitoring the arterial blood pressure and some arbitrary vital signs, for example, heart rate and pulse oxymetry. The bracelet 1, inside its enclosure, has various parts that will be described below. On its surface it carries several external parts, such as inflatable bladder 13 and optical elements 23 of the medical detector 21. The bladder 13 is for measuring arterial blood pressure (BP).

Air pressure inside the bladder 13 is controlled by the air pump 14 and air valve 17 and measured by the air pressure sensor 16. All these components are interconnected by the pneumatic tubing 15. These components are typical for any conventional arterial blood pressure monitor known in the art and not described here in detail. It is important to note that bladder 13 generally circumferences wrist 9 to compress its internal artery 10 on a command from the controller 18. The compressing air pressure should vary between somewhat below the diastolic pressure (DIA) and above the systolic pressure (SYS). A maximum air pressure must not exceed 350 mmHg which may require an addition of a safety valve (not shown) attached to the tubing 15. The pressure-related components are interfaced with the controller 18 that turns on and off the drivers (not shown) for pump 14 and valve 17. It also monitors air pressure via the pressure sensor 16 and converts pressure signal to a digital format. The results of monitoring are fed to the near-range wireless communications module 19 that transmits and receives radio signal 7.

The bracelet components are powered by a primary or rechargeable battery 20. The controller 18 takes the BP and other vital signs either on its own timing or on command received from the smartphone 2 via the module 19. Generally, information transmitted by module 19 contains only conditioned signals from the sensors and not the actually computed diastolic and systolic pressure numbers. These are preferably computed by the phone 2 microprocessor an accordance with the installed app. This allows future modifications and updates of the phone app without changing hardware or software of the bracelet 1.

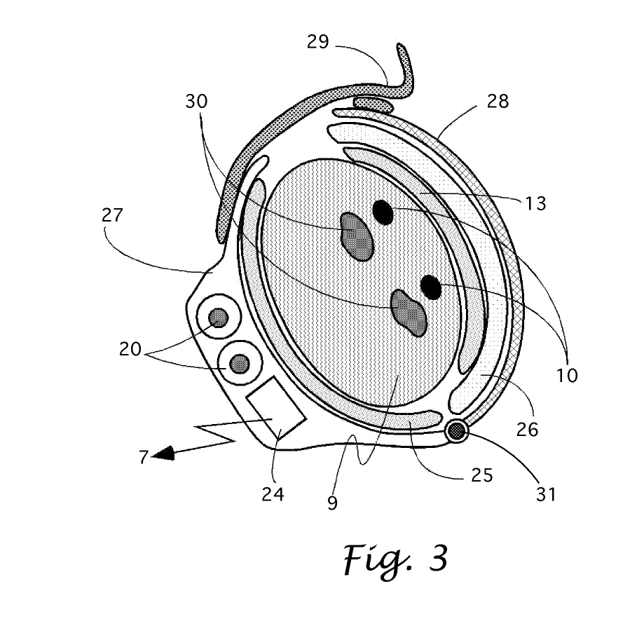

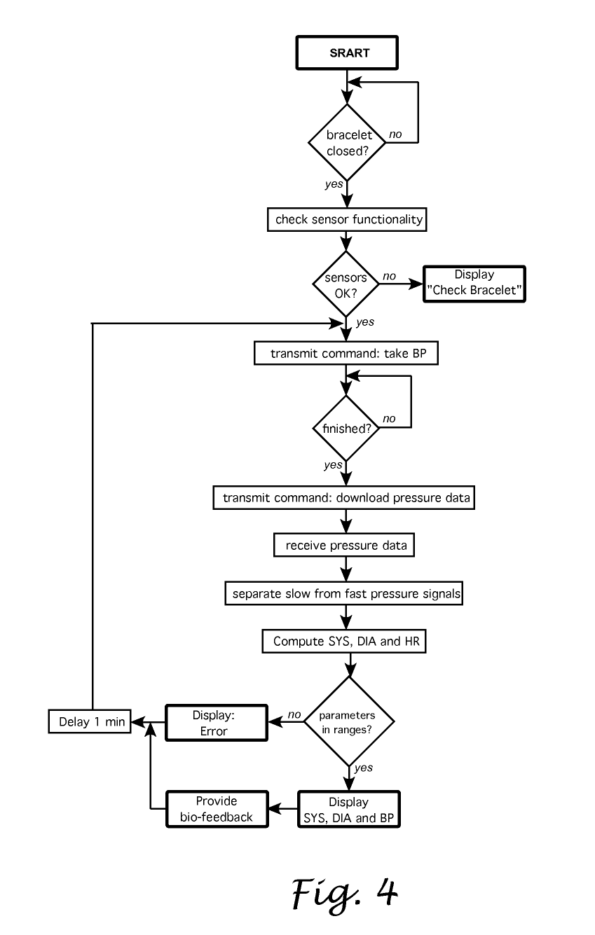

To better illustrate a mutual disposition of the components, FIG. 3 shows a cross-sectional view of the patient's wrist with the attached bracelet. The bracelet is comprised of two half-shells: the front shell 28 and the back shell 27. The shells can move relative to one another by rotating around the pivot 31. The front shell 28 supports bladder 13. The soft cushions 25 and 26 may be added on the insides of the half-shells for a better patient comfort. Before placement on a wrist, the front shell 28 is rotated on pivot 31 to open the bracelet for positioning on the wrist 9. Then the front shell 28 is closed and locked to the back shell 27 in a fixed position by a suitable locking device, such as Velcro tape 29. This makes the bracelet clearance adjustable for a particular wrist size.

Bladder 13 may be inflated by pump 14 to compress arteries 10 against the supporting bones 30 inside the wrist 9, causing a restriction of the blood flow inside the arteries. The blood flow restriction results in mechanical arterial oscillations that are detected by the pressure sensor 17 and will be interpreted by an app in a smartphone 2 to compute the arterial blood pressure according to one of the algorithms known in the art. Various electrical and mechanical components are positioned inside the front and back shells 28 and 27, respectively. This is illustrated by the battery 20 and an electronic module 24 that contains most of the components embraced by the dotted line in FIG. 2.

Besides the arterial blood pressure, other vital signs can be monitored by the system of a bracelet 1 and phone 2. For example, heart rate and its variations can be directly derived from the fast changing component of the pressure signal received from the pressure sensor 16. Other vital signs may be obtained by an additional medical detector 21 (see FIG. 2) that interfaces with the wrist 9. One example of the detector 21 is a pulse oximeter that contains optical elements 23. Typically, these optical elements comprise a light detector and two LEDs: red and infrared. Design and functionality of a pulse oximeter are well known in art and thus not described here. Other examples are bio-impedance and chemical composition of sweat and blood. Note that all bracelet components related to sensing of vital signs are physically coupled to the patient body or body part surface. The coupling my be direct by touching or by intermediate media, such as air, e.g.

Operation of the devices according to the present invention can be outlined as follows. The patient snaps on the bracelet 1 on her wrist and latches it for a comfortable wearing by a latch 6 or Velcro tape 29. Indicating light 22 shows that the bracelet is in a correctly secured position and power is turned on. The bracelet 1 establishes a wireless communication with the smartphone 2 that initiates the monitoring application (app) that was pre-loaded into the phone 2 memory. After a routine self-check, the phone 2 sends a wireless command to controller 18 to take a blood pressure. The pump is inflated, then deflated according to one of a predetermined algorithms will known in the art. The output signals from the pressure sensor 16 are digitized and transmitted to the phone 2 where the app computes the systolic, diastolic and mean pressures and also calculated a heart rate, RR-interval variability and other cardiac parameters. FIG. 4 illustrates a simplified flow-chart of the app for measuring blood pressure (BP). It is seen that the bracelet and smartphone work together in concert: the phone 2 sends commands to the bracelet, then receives and processes biomedical signals to compute and display the SYS, DIA pressures and HR. After the BP is measured, the bladder 13 is deflated and blood flow via artery 10 is restored, pulse oxymetry data are optically measured by the detector 21 and also transmitted to the phone 2 that computes percentage of the hemoglobin oxygenation. The results of the vital signs monitoring are treated by the phone 2 according to the app, for example, they may be plotted, alarmed, transmitted to a medical office, stored for future retrieving, etc.

An important feature of this invention is that the smartphone 2 can in real time provide via its output means (display and/or speaker) a biofeedback information to the patient in accordance with the monitored biomedical signals and pre-defined algorithm programmed into the app. For example, if the device is used in fitness, the HR and BP numbers can provide guidance to the strength and duration of the exercise procedure.

While the invention has been particularly shown and described with reference to a number of preferred embodiments thereof, it will be understood by those skilled in the art that various changes in form and details may be made therein without departing from the spirit and scope of the invention.

1. Biomedical monitor for a patient, comprising a sensing device and a mobile communication device, where
the sensing device is detached from the mobile communication device and physically coupled to a part of the patient body and contains a near-range communication module for a wireless exchange of signals between the sensing device and the mobile communication device and further contains at least one detector of a vital sign of the patient;
the mobile communication device comprises an output means and a memory with a pre-loaded software application for commanding the sensing device and processing signals received from the sensing device and further presenting results of such processing on the output means in accordance with the rules pre-defined in the software application.

2. Biomedical monitor of claim 1 where the sensing device is configured to have a shape of a bracelet being adapted for attachment to a wrist or ankle of the patient.

3. Biomedical monitor of claim 1 where the sensing device further comprises an inflatable bladder fabricated of a pliant material, air pump, air valve and air pressure sensor for measuring pressure in the bladder.

4. Biomedical monitor of claim 1 where the sensing device further comprises a second detector of a vital sign, such vital sign being one of the group of a heart rate, blood oxymetry, chemical composition, and body impedance.

5. Method of monitoring of a patient vital sign comprising the steps of:
providing a sensing device wearable on a part of a patient body and a wireless communication device having an output means and being positioned on or near the patient;
Incorporating into the sensing device a near-range wireless communication module for exchange of signals with the wireless communication device, an electronic module and at least one sensor of the patient vital sign;
installing into the wireless communication device an applications software for controlling the sensing device and processing the patient vital sign;
attaching said sensing device to the patient body for physical coupling the sensor and the patient body part;
measuring at least one vital sign and generating by the electronic module a first signal,
transmitting the first signal through the wireless communication module;
receiving and processing the first signal by the wireless communication device, and
sending the processed signal to the output means.

6. Method of monitoring of patient vital signs of claim 5 where said one vital sign is arterial blood pressure.

7. Method of monitoring of patient vital signs of claim 5 where said processing includes actuation of an alarm if the vital sign exceeds limits predefined in said application software.

8. Method of monitoring of patient vital signs of claim 5 where said application software sends a bio-feedback information to the output means in accordance with pre-defined rules.

9. A mobile communications device comprising a near-range wireless communication module and a memory adapted for storing a software application and being positioned in proximity to an external device being attached to a patient, wherein
the software application is adapted for controlling the external device and processing medical signals received by the near-range communication module from the external device;
the external device is adapted for collecting and conditioning medical signals from the patient body surface.

10. A mobile communications device of claim 9, wherein said external device is positioned at distance no greater than 2 m from the mobile communication device.

11. A mobile communications device of claim 9, wherein the external device comprises an inflatable cuff for compressing an artery of a patient.

12. A mobile communications device of claim 9, wherein the external device is configured for being positioned on a patient limb.

13. A mobile communications device of claim 9, wherein the medical signals are selected from the group of arterial blood pressure, blood oxymetry, heart rate, heart rate variability and chemical composition.
